(19) United States
(12) Patent Application Publication
(10) Pub. No.: US 2019/0254744 A1
(43) Pub. Date: Aug. 22, 2019

(54) Handpiece Apparatus, System, and Method for Laser Treatment

(71) Applicant: Berkshire Photonics LLC, Washington Depot, CT (US)

(72) Inventors: Jeffrey Miller, Bethlehem, CT (US); Michael Stuart William Windebank, Torrington, CT (US)

(73) Assignee: Berkshire Photonics LLC, Washington Depot, CT (US)

(21) Appl. No.: 16/279,343

(22) Filed: Feb. 19, 2019

Related U.S. Application Data
(60) Provisional application No. 62/632,675, filed on Feb. 20, 2018.

(51) Int. Cl. A61B 18/20; A61B 18/22

(52) U.S. Cl. CPC A61B 18/203; A61B 2018/00922; A61B 18/22

(57) ABSTRACT

A handpiece for laser treatment of a target surface on a patient, such as a skin surface, mitigates the effects of backscattered radiation at high optical power levels that can heat the handpiece to dangerous levels, as well as exposing practitioners and patients to the backscattered light and/or the handpiece structures heated thereby. A standoff affixed to the handpiece is configured to guide the handpiece position with respect to the target surface during use, such that the angle of incidence of treatment light is held near a predetermined angle that is not perpendicular to the target surface. This angle decreases the fraction of treatment light scattered directly back toward the handpiece, and the standoff may additionally be used to set a preferred distance to the target surface. Systems for laser treatment using a handpiece having these features, and methods for laser treatment using the handpiece are also provided.

### BACKGROUND OF THE INVENTION

### 1. Field of the Invention

The present invention relates generally to the field of treating tissue with electromagnetic radiation, and more particularly, to medical handpieces and related systems and methods for laser treatment that reduce the scattering of radiation back toward the handpiece.

### 2. Description of the Related Art

High-power lasers and similar high-intensity light sources are being used for an increasing number of therapeutic applications as well as medical surgical procedures. Many types and wavelengths of lasers are used for different applications, from dermatological to surgical. In many of the therapeutic applications, photochemical effects that stimulate biological processes are thought to be involved, as opposed to surgical applications that use primarily photothermal effects. For example, laser treatment is increasingly being applied to promoting wound healing, scar tissue reduction, and treatment of different types of musculoskeletal and nerve pain caused by various disorders and injuries. Often, near-infrared (NIR) wavelengths are chosen for these photochemical therapies, typically using wavelengths ranging from about 800 to 1000 nm, which achieve a beneficial combination of penetration into deeper layers of tissue (due to lower tissue absorption and scattering at these wavelengths) and the targeted stimulation of particular biochemical processes. Such NIR wavelength radiation is often efficiently generated using high-power diode lasers, available at wavelengths such as 808-810 nm and 976-980 nm, and may be delivered through flexible fiber optics to a treatment handpiece that is used to direct the radiation to the skin surface or body part that is to be treated. For many of these treatments, it is desirable for the handpiece to irradiate an area that is several square centimeters in size so as to efficiently cover an extended target zone in a minimum time, unlike the requirements for surgical laser handpieces, that often focus laser power to a very small spot to accomplish cutting or cauterization. Such treatments are used in both human and veterinary applications, with large animals such as horses sometimes requiring higher dosages (exposures) of radiation to achieve the desired therapeutic results.

Exposures for different treatments vary, as well as the optimum irradiance level during the treatment. Deeper tissues may require higher irradiances to deliver the optimum irradiance to the depth to be treated. Examples of total exposures required per treatment, in units of total deposited radiative energy in joules (J), for enhancing and accelerating wound healing can range from 5000-10000 J, whereas required exposures for treatment of large joints in equine patients can range even higher, from 6000-50000 J. [References for total exposure include the following files: physician-details-diowave-laser-2015.pdf and veterinary-brochure-pdf.pdf, downloaded Jan. 30, 2018 from www.diowavelaser.com] The use of higher-power lasers enables shorter exposure times to achieve the same dosage (total deposited energy), saving time and money. For example, a 60 W laser can deliver 3600 J/min. However, not all this power is delivered to the target zone, which can be several mm or even cm beneath the skin surface. This is due to optical reflection, scattering, and absorption in the skin and other structures between the surface and the target zone, which can attenuate the NIR radiation by a significant factor of 3 to 10 or more. Several minutes of laser irradiation of the skin may thus be needed to achieve the desired high exposures for a particular treatment session. The same handpiece may be used all day long for sessions with multiple patients, and excess heat can build up within the handpiece.

It has been found that during the extended use of a laser handpiece at high optical power levels, the optics and internal structures of the handpiece can heat up to dangerous temperatures. This heating of the handpiece is exacerbated by the reflective and scattering properties of human skin, which can have 35-70% reflectivity with a significant specular component. When used in close proximity to a target surface that is a skin surface, and approximately perpendicular to the surface, as is common practice, the specular component as well as the forward diffuse component of the scattering are directed back into the handpiece, thus causing a large fraction of the high-power radiation emitted from the handpiece to reflect or scatter directly back into the handpiece, resulting in heating, caused by the absorption therein of high-power laser light. The resulting temperatures can be high enough to injure patients or practitioners by contact with structures at the emitting (distal) end of the handpiece that are thermally connected to the internal structures heated by back-reflected light.

Previous laser treatment or surgical handpieces have sometimes incorporated features that aid in maintaining a fixed spacing between the handpiece and a target surface in order to maintain either a consistent irradiated area (e.g. for shaped treatment areas) or a tightly focused spot (e.g. for cutting or surgery). But no known prior art has recognized a need for guiding a handpiece at an orientation other than “vertical,” i.e., to direct the treatment beam at an angle other than perpendicular to the target surface.

There is accordingly a need for handpiece designs, laser treatment systems, and treatment methods that reduce the causes, and minimize the effects of backscattered radiation causing undesirable heating on patients and practitioners, and on treatment equipment such as the handpiece itself.

### BRIEF DESCRIPTION OF THE SEVERAL VIEWS OF THE DRAWINGS

For a more complete understanding of the present invention, and the advantages thereof, reference is now made to the following description taken in conjunction with the accompanying drawings, in which:

FIGS. 1A through 1D are schematic block diagrams depicting systems for laser treatment according to three embodiments of the present invention;

FIGS. 2A and 2B show two views of a laser handpiece assembly according to an embodiment of the present invention;

FIGS. 3A and 3B show side views of laser handpieces according to two different embodiments of the present invention having different angles of incidence;

FIG. 4A is a view of a laser handpiece according to an embodiment of the present invention;

FIG. 4B depicts a cross-sectional view of the laser handpiece shown in FIG. 4A;

FIGS. 5A and 5B are views of laser handpieces having different angles of incidence according to two different embodiments of the present invention, from a direction perpendicular to the treatment surface, each showing an optical beam and a projected spot on a target surface;

FIGS. 6A and 6B are two additional views depicting a laser handpiece assembly according to an embodiment of the present invention;

FIG. 7 is a perspective line drawing of a system for laser treatment according to an embodiment of the present invention;

FIG. 8 is a side view showing a laser handpiece in use during the practice of a method for laser treatment according to an embodiment of the present invention; and

FIG. 9 is a flow chart illustrating a method for laser treatment according to an embodiment of the present invention.

### DETAILED DESCRIPTION

While the making and using of various embodiments of the present invention are discussed in detail below, it should be appreciated that the present invention provides applicable inventive concepts that can be embodied in a wide variety of specific contexts. The specific embodiments discussed herein are merely illustrative of specific ways to make and use the invention and do not delimit the scope of the invention.

Various embodiments are described below of the present invention that includes treatment handpieces, systems, and methods of use, having features designed to reduce the backscattering and/or back-reflection of high-power light beams, such as laser beams, in the direction back toward the treatment handpiece. Reduction of the amount of high-power laser light that is directed back into or onto the handpiece serves to greatly reduce exposure to scattered light that can cause undesirable overheating of the handpiece, its parts, and related structures that may come into injurious contact with patients or practitioners. Inventive features of the various embodiments also help in reducing the exposure of practitioners holding the handpiece to excessive levels of the treatment light.

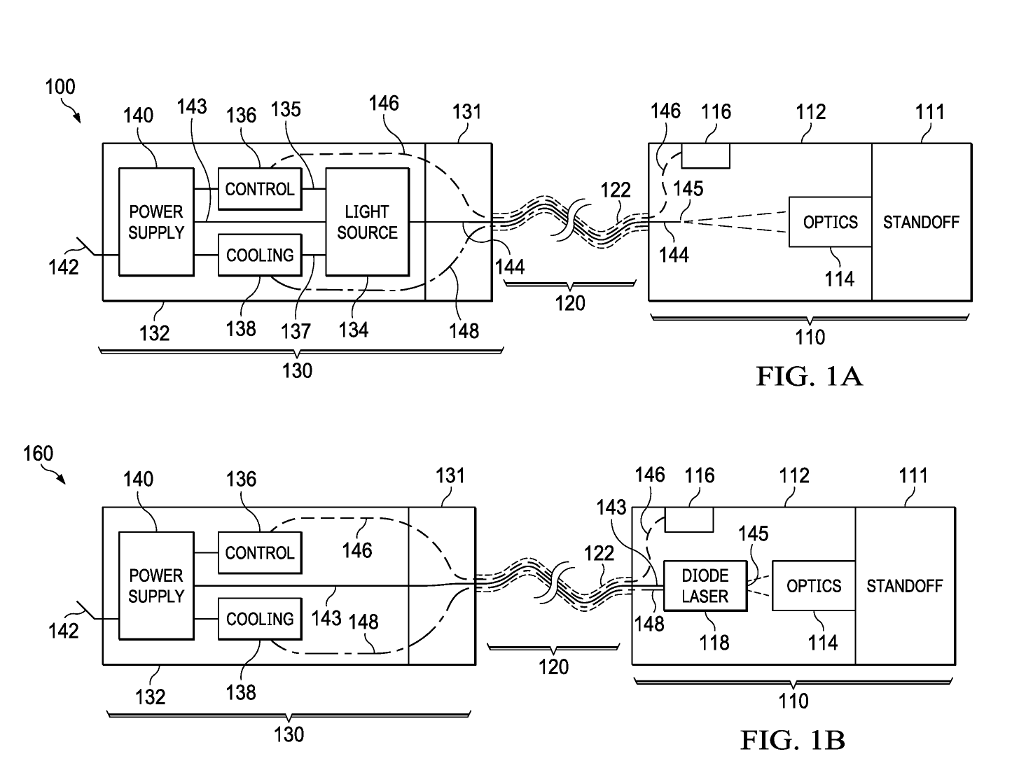

Referring now to FIG. 1A, a schematic block diagram depicting a system for laser treatment 100 according to one embodiment of the present invention is shown. The system comprises a handpiece 110 for directing electromagnetic radiation, typically high-powered laser light supplied by a light source 134, under control of a treatment practitioner to a surface or area of a patient to be treated; an umbilical 120 for supplying electromagnetic radiation and/or other signals to the handpiece 110; and a system controller 130. Besides any of a number of types of laser, light sources 134 used in conjunction with system 100 can alternatively comprise one or more light emitting diodes (LEDs), flashlamps, or other source of optical electromagnetic radiation having wavelengths and power levels suitable for different therapies or surgeries. Handpiece 110 has a housing 112, which in some embodiments can be in the form of an ergonomic handle that serves as an interface for practitioner to manually control the exposure as well as to direct the laser light. Handpiece 110 supports optics 114 for shaping and directing laser light to the surface to be treated, as well as an optional trigger 116 by means of which the practitioner can turn the treatment laser light on and off as desired. Laser light emanates from an emitting aperture 145 within housing 112 and is transmitted through optics 114. In the embodiment of FIG. 1A, emitting aperture 145 is defined by the exit end face of an optical fiber 144. Optics 114 can include lenses, mirrors, windows, and/or optical fibers, and can optionally be omitted if an optical beam generated in the handpiece 110 does not require further protection or conditioning before emission to the patient surface. More specifically, optics 114 can comprise any combination of free-space propagation or divergence, mirrors, lenses, windows, and/or optical waveguides used to form, direct, shape, focus, and/or defocus an optical treatment beam (not shown) so as to irradiate a target surface (also not shown) that is to be treated. In some embodiments, optics 114 can include means for scanning the treatment beam in a pattern on the target surface, and/or means for shuttering the beam to block it intermittently between or during treatments. Optics 114 can be supported directly by features in housing 112, or may be held in position and alignment by a separate optics holder (not shown) supported within housing 112, as will be described later with respect to a specific embodiment.

The function of the optional trigger 116 may be replaced by a control or button on system controller 130, a timer control, or a floor-mounted switch such as a footswitch or foot pedal (not shown) in some embodiments, as is known to those skilled in the art, with no change to the scope or spirit of the present invention. A finger-operated trigger switch located at handpiece 110 on housing 112 to initiate laser irradiation to perform the function of trigger 116 has been found to be safe and convenient for practitioners and patients, and embodiments depicting this are shown in the accompanying drawings. The position of such a finger-operated switch can be varied for use with different fingers and/or for ergonomic reasons, as will be discussed later.

As discussed above, backscattering or back-reflection of high-power laser light from the surface to be treated has been found in some cases to cause dangerous heating of some parts of handpiece 110. To mitigate this effect, a key feature in some embodiments is a standoff 111 incorporated into or affixed onto handpiece 110 to aid the practitioner (user) in setting, during irradiation of the patient, the angle of incidence of the treatment light beam on the target surface, the distance of the handpiece to the surface, or preferably both distance and angle. Standoff 111 is used as a guide to position handpiece 110 during operation. Exemplary structures and arrangements for the standoff 111 will be described in more detail below.

Other optional features can be built into or onto handpiece 110 that may not be shown in all of the accompanying drawings. These optional features can include any or all of the following, but are not limited to: mechanical or optical alignment/pointing aids to more precisely indicate the area to be treated, such as mechanical features on standoff 111, or a light source for an aiming beam as described below; sensors for measuring temperature on or within the handpiece 110 or for taking temperature readings on the patient surface; sensors for measuring parameters of the target surface such as surface color or reflectivity; displays for parameters such as light source settings, e.g. on/off status or pulse and power parameters, or other handpiece or system status indicators; means for cooling the handpiece 110 or patient surface during treatment; protective optics covers; and mechanical features to facilitate hanging or storage, mounting on a machine or stand, or cleaning.

Handpiece 110 can be connected to a system controller 130 that provides power and other control functions for use with the handpiece 110. For convenience in the manual positioning and directing of treatment light, handpiece 110 can be connected to system controller using a flexible or articulated connection, shown here as a flexible umbilical 120 that can contain one or more flexible optical fibers, electrical cables, and/or cooling tubes, pipes, or other connections. Umbilical 120 can include a protective jacket 122 such as an armored casing around cables, tubes and signal wires routed through the umbilical, and the protective jacket 122 may in some embodiments have additional smooth flexible coatings to facilitate cleaning or disinfection of umbilical 120. Umbilical 120 connects to system controller 130 through an interface 131 that can be permanently affixed, or preferably, for convenience in replacing handpieces or changing handpiece types, can contain demountable connectors (not shown) for optical fibers, electrical cables, and/or cooling connections. Thus umbilical 120 and any cables, wires, and/or optical fibers or waveguides therein may be detachable from system controller 130. Alternatively, in some embodiments, umbilical 120 may be detachable only from handpiece 110, or detachable at both ends from both system controller 130 and handpiece 110. Interface 131 can further include safety devices or proprietary features such as identification or verification means (not shown), e.g. a radio-frequency identification (RFID) device for a connected handpiece 110 that can be used to indicate to the system controller 130 what type of handpiece is in use, to limit or prevent reuse of used handpieces, to enforce the use of only authorized handpieces, or to support and enforce handpiece cleaning or disinfection protocols or optical fiber maintenance. These safety devices or proprietary features can be incorporated e.g. into a demountable connector or connectors in interface 131, or (not shown) into handpiece 110, and can operate in concert with an electronic processor e.g. within controller 136 that can reside either in system controller 130 as shown, or, in some embodiments, within handpiece 110.

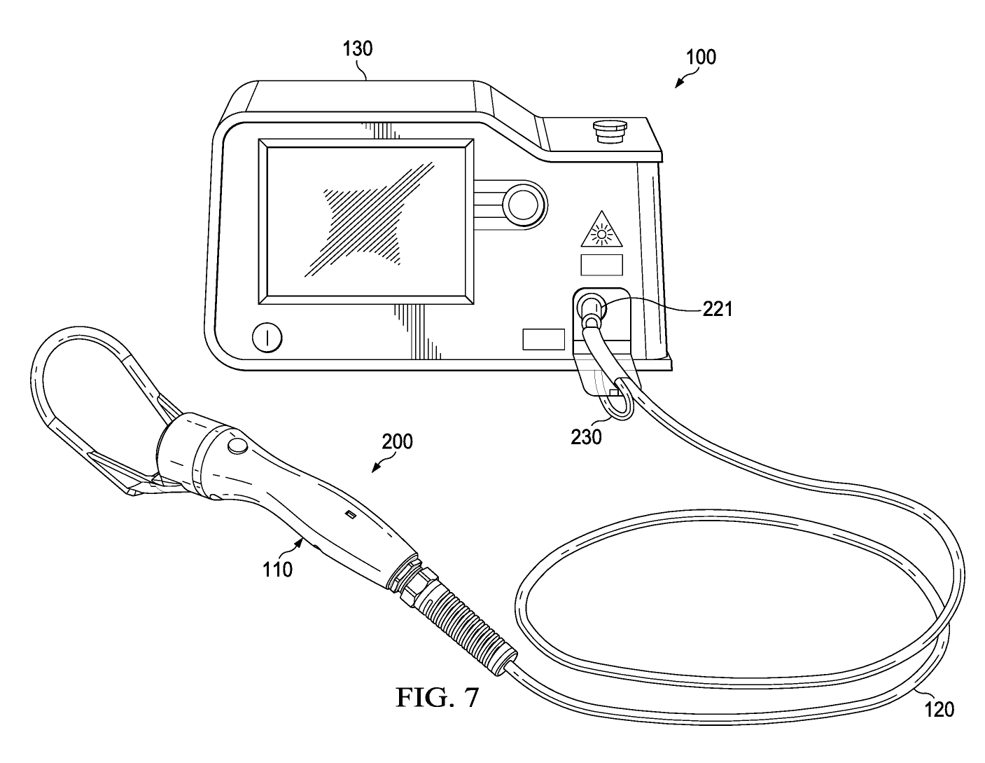

System controller 130 is optionally configured such that components to perform its functions, to be described presently, are housed in a separate enclosure 132 from handpiece 110. This configuration for laser treatment system 100, which allows for a lightweight and separable handpiece 110, is illustrated in FIG. 7. Again, referring to FIG. 1A, in one embodiment light source 134, which can be a high-power laser such as a near-infrared diode laser, is housed within enclosure 132 of the system controller 130. Other types of light source 134 are possible, such as halogen lamps or high-power flashlamps, diode-pumped or flashlamp-pumped solid-state lasers. In the embodiment illustrated in FIG. 1A, light source 134 emits treatment light that is transmitted through optical fiber 144 to optics 114 in handpiece 110. Light source 134 can also include an additional separate visible light source (not shown) to generate an aiming beam or pilot beam that is also transmitted through optical fiber 144 and directed through optics 114 to the treatment surface, to indicate to the user the location where the treatment beam, which may be invisible, will next be incident on the patient should trigger 116 be activated. A separate aiming beam (not shown) that is not transmitted through optical fiber 144 can also be provided in handpiece 110. Electrical power for light source 134 is provided through power cable 143, and control of light source 134 is communicated by the electrical connection 135 from controller 136. Controller 136 can include a driver for the light source, e.g. to provide and condition electrical current to a laser diode; circuitry or processors for setting light source parameters such as optical power output level and pulse timing parameters such as pulse length, repetition rate, and counters for number of pulses in a given exposure period; processors for inputting and storing user inputs for setting such parameters and remembering a number of stored treatment profiles or protocols; and one or more input controls and a display or displays, comprising a user interface for interacting with the system for laser treatment 100 and making and displaying its settings and status. Controller 136 can be constructed using discrete or integrated electronics or processors and can also interact with a safety device or verification means, as described in the previous paragraph, to implement its function and to prevent operation of light source 134 as called for. A cooling system 138 connected to light source 134 through cooling connection 137 can be provided to remove excess heat from light source 134 during high average power operation. Cooling system 138 can use any refrigeration technology or combination thereof, including liquid chillers, thermoelectric cooling, and fans or free convection or forced-air cooling systems. Cooling connection 137 can comprise gas or liquid cooling channels such as piping or tubing, and/or air flow. Cooling system 138 and cooling connection 137 can optionally reside in handpiece 110 in order to cool light source 134 or components thereof in embodiments in which light source 134 is in handpiece 110. Additional cooling systems and/or cooling connections can be provided to cool structures within the handpiece 110, such as optics 114, or housing 112, or standoff 111, or even to cool the patient. Electrical power for system 100 can be derived from a mains connection 142 including a wall plug (not shown) and mains power cable, and converted into usable voltages and currents by power supply 140. Optionally, power supply 140 can comprise an electrical battery or batteries (not shown) that can be rechargeable, or replaceable and completely separated from mains power, eliminating the need for a mains connection 142.

In the embodiment shown in FIG. 1A, treatment light is supplied from light source 134 in system controller 130 to handpiece 110 through umbilical 120. Specifically, in a flexible umbilical as illustrated, an optical fiber 144 running through umbilical 120 is used to conduct light from light source 134 to handpiece 110. An electrical cable 146 can also run through the umbilical 120 to provide electrical connections to the handpiece 110 conveying electrical signals in either direction, including a signal from trigger 116 back to controller 136 in this embodiment. Optional cooling as described in the previous paragraph can be supplied to handpiece 110 through a cooling connection 148 that can also be conducted through umbilical 120, for applications requiring cooling of the handpiece 110 or the optics 114 or standoff 111 therein. Such optional cooling arrangements within handpiece 110 are not shown in FIG. 1A. Cooling connection 148 can comprise one or more fluid channels or electrical connections, as will be detailed later with respect to the embodiment shown in FIG. 1D.

A schematic block diagram of a second embodiment for a system for laser treatment 160 is shown in FIG. 1B. This embodiment differs from the embodiment described in conjunction with FIG. 1A in that light source 134 is removed from system controller 130 and takes the form of a laser (in this case a diode laser 118) supported in housing 112 within handpiece 110. Treatment light emanates from emitting aperture 145 on diode laser 118 and is shaped and directed by optics 114. In this embodiment, no optical fiber connecting system controller 130 to handpiece 110 is required. Instead, electrical cable 146 is used to bring power and control signals to and from controller 136 to diode laser 118 within handpiece 110, as well as to return signals (which may include signals such as a control signal from trigger 116) to system controller 130 from handpiece 110. Cooling connection 148 can conduct cooling fluids from cooling system 138 through umbilical 120 to handpiece 110 in order to cool diode laser 118 as shown. Some functions of system controller 130 such as cooling system 138 and/or power supply 140 may alternatively be housed together in handpiece 110, reducing or eliminating the need for umbilical 120 and even a separate system controller 130, but at the current state of the art in laser efficiencies and power levels, a cooling connection 148 from system controller 130 is likely necessary.

In some embodiments, the use of a highly power-efficient light source, such as a diode laser 119 as in FIG. 1B, may permit a single-piece solution, in which handpiece 110 can incorporate all the features of system controller 130. In such a system, umbilical 120 connecting system controller to 130 can be eliminated, or its functions incorporated entirely internally to handpiece 110. Handpiece 110 can be powered from a mains electrical source through its own mains connection 142; or in some embodiments (not shown), electrical power can be provided from batteries housed entirely within handpiece 110, possibly eliminating the need for a mains connection 142. Such batteries, if used, may be replaceable or rechargeable as is known in the art.

In FIG. 1C, a third embodiment of a system for laser treatment 170 is shown, in which an optically-pumped laser such as a diode-pumped solid-state laser (DPSS laser) 119 is housed in handpiece 110. In this embodiment, power, control, and cooling systems are housed in system controller 130, but instead of a treatment light source, a pump light source 174 is housed in system controller 130. Pump light source 174 can be, for example, a diode laser emitting at a “pump wavelength,” i.e. a wavelength that is strongly absorbed by pump bands of a solid-state laser crystal. Light from pump light source 174 is conducted through optical fiber 144 into handpiece 110 and therein used to energize (“pump”) a crystal or glass element doped with lasing ions in the DPSS laser 119 that generates the treatment light. In this embodiment, rather than conducting light at the treatment wavelength, optical fiber 144 conducts light at the pump wavelength to the handpiece 110. Light generated by DPSS laser 119 emanates from emitting aperture 145, and is conditioned and directed by optics 114 toward the target surface to be treated.

FIG. 1D is a variation of FIG. 1A showing an embodiment in which cooling connection 148 is extended to standoff 111 to cool the standoff 111, and/or a target surface on the patient in contact with standoff 111. Cooling connection 148 can consist of fluid channels conducting a liquid or gas fluid cooled by cooling system 138, to remove heat from standoff 111. Cooling connection 148 can comprise both supply and return cooling channels for a closed-loop cooling system where heat is removed from return channel in system 138 and sent back to handpiece 110. Alternatively, a cooled fluid such as a gas or liquid can be conducted from cooling system 138 to be sprayed onto an area of the patient to be cooled by forced convection during laser irradiation. Structures in or on standoff 111 can be used to direct such a spray. In yet another embodiment of a cooling device, cooling connection 148, instead of containing fluid connections, can comprise electrical connections used to power one or more Peltier-type thermoelectric cooling devices in or on standoff 111 to cool the patient's target surface through heat conduction. A cooling plate (not shown) that is transparent to the laser light can be used to conduct heat away from the target surface on the patient for cooling. Whether fluid or electrical cooling is used, cooling connection 148 can further comprise electrical connections to conduct temperature signals from sensors in or on standoff 111 or elsewhere in or on handpiece 110 for temperature measurement and/or control.

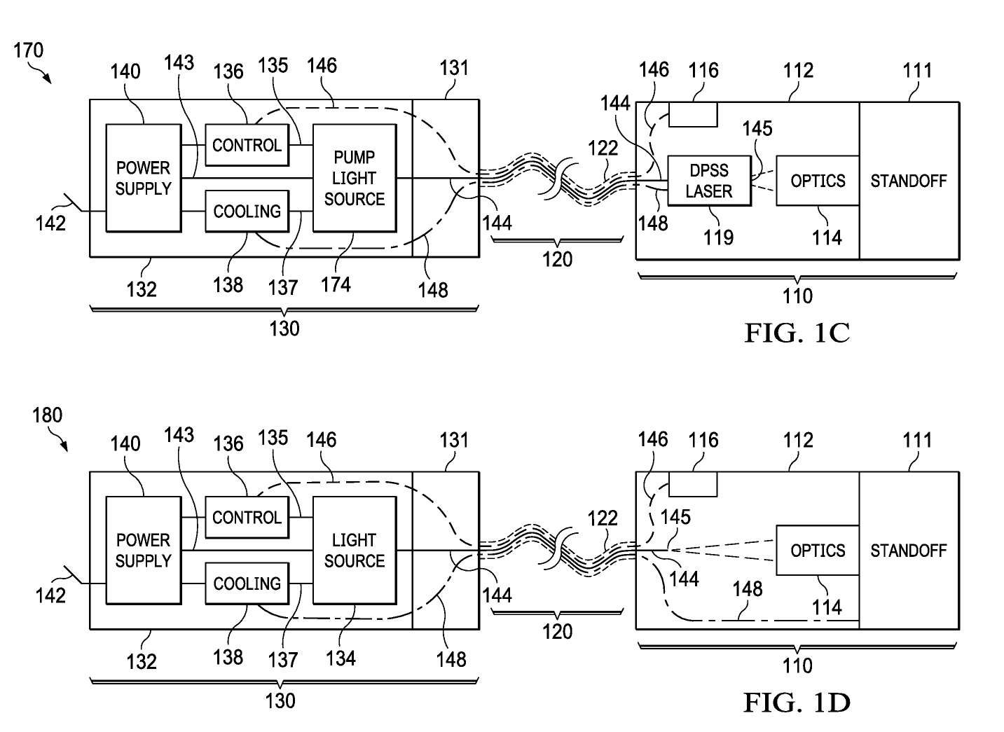

Referring now to FIGS. 2A and 2B, two views of an exemplary laser handpiece assembly 200 according to an embodiment of the present invention are shown.

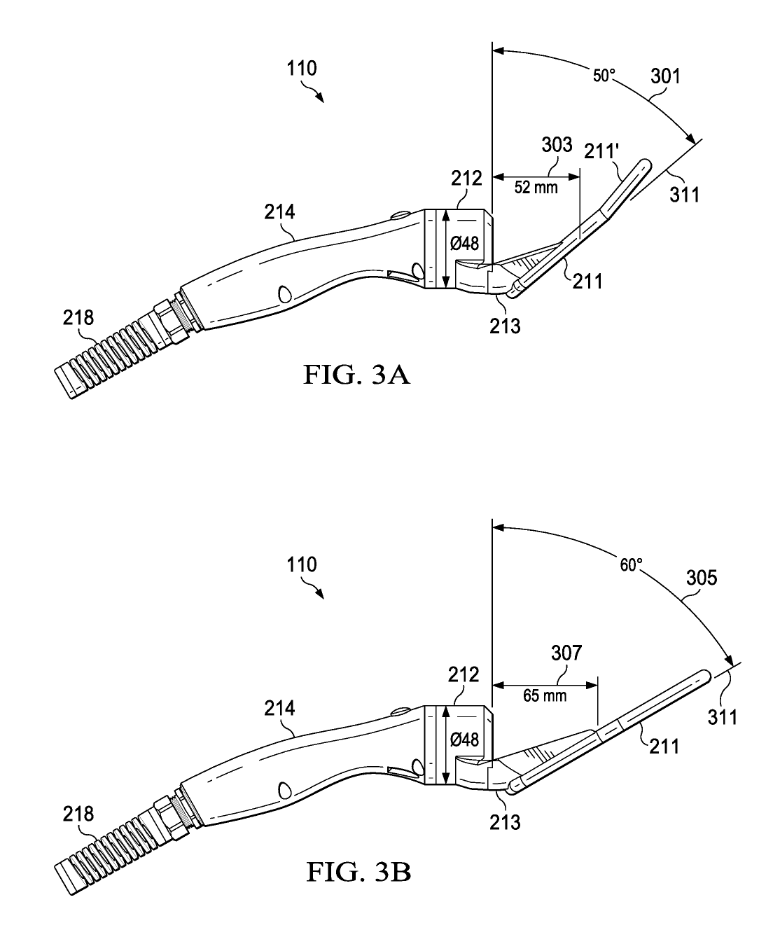

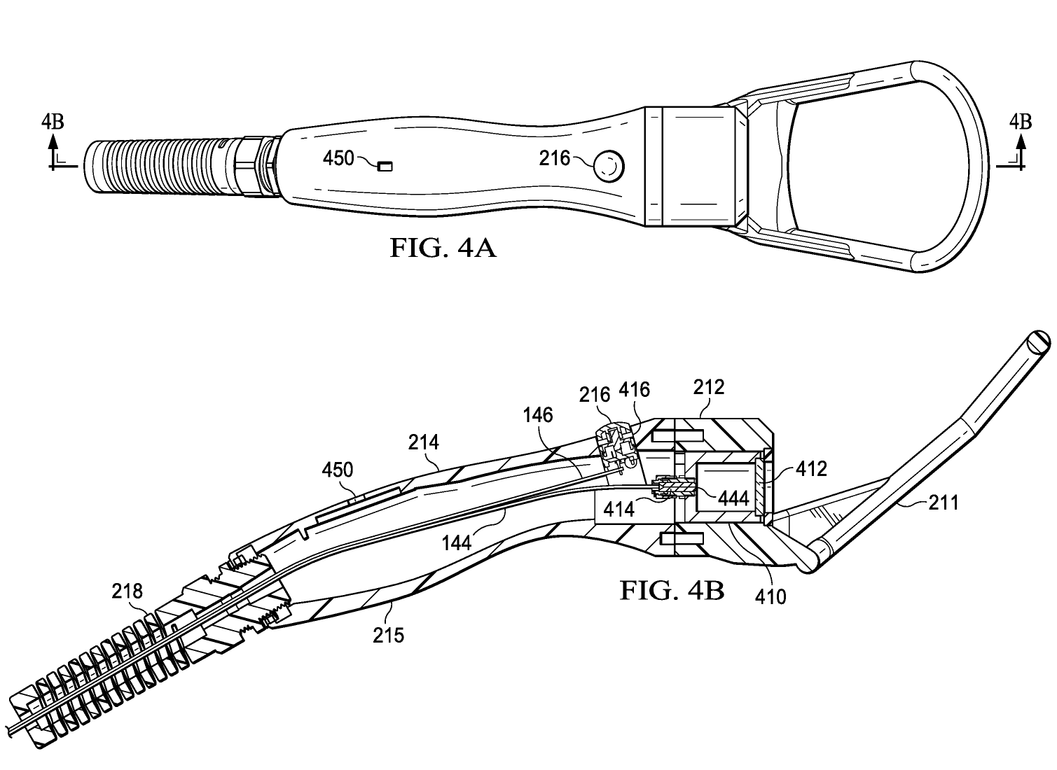

Referring specifically to FIG. 2A, handpiece assembly 200 comprises handpiece 110 that in turn includes a standoff 111 connected to housing 112, as well as umbilical 120 and associated parts for strain relief and interconnection. Housing 112 includes a handle portion 214 preferably ergonomically shaped for ease in holding and manipulation by a human hand and having a section shaped as a grip 215 as well as optional trigger button 216 placed opposite grip 215 to facilitate operation of the trigger button using a thumb. Handle portion 214 supports the distal portion 212, and in this embodiment, standoff 111 comprises a structure in the form of a loop-shaped bail 211 connected to and supported by attachment 213 to distal portion 212. During use, holding handpiece 110 in a position and orientation with respect to a target surface such that bail 211 is in contact or near contact over much of its perimeter with the target surface helps a user to ensure that the angle of the treatment laser beam and its spot size (by controlling distance of distal portion 212 from the target surface) are close to optimal for treatment and for prevention of back-reflection. Attachment 213 can be fixed to permanently connect bail 211 to distal portion 212 (or it may attach bail 211 to another portion of housing 112), or attachment 213 can be configured to allow bail 211 to be removable from handpiece 110, i.e. to facilitate cleaning. In order to preserve alignment and to discourage users from attempting to defeat the protective function of standoff 111, it is preferable that bail 211 be permanently connected through attachment 213 to housing 112. In this drawing FIG. 2A and others, the attachment area connecting bail 211 to distal portion 212 is shown using an exemplary monolithic, fixed attachment 213 formed integrally with distal portion 212 and bail 211. As will be seen later with reference to FIG. 4B, distal portion 212 housing 112 can also support and enclose, either directly or indirectly, at least a portion of the internal optics 114 (not shown in FIG. 2A).

Referring now to FIG. 2B, a view of handpiece assembly 200 as in FIG. 2A is seen looking directly toward the distal portion 212 along the optical axis, showing optical aperture 234 from which the optical beam emanates. It will be seen shortly how bail 211 and attachment 213 assist in maintaining a target surface (not shown) a predetermined distance and angle from optical aperture 234 during operation. Referring again to FIG. 2A, other parts of handpiece assembly 200 in this embodiment that are related to the umbilical 120 and its connections include the following: strain relief 218 used to support umbilical 120 where it is attached to handpiece 110; fiber optic connector 221 to terminate the end of optical fiber 144 and form the optical portion of interface 131; and electrical cable 230 and associated electrical connector 231 used to form the electrical portion of interface 131, connecting a laser on/off signal controlled by trigger button 216 to system controller 130. Electrical cable 230 can have multiple conductors in order to perform one or more functions. In some embodiments, electrical cable 230 can be used to conduct electrical power to handpiece 110 for various functions such as indicators, and/or to carry sensor or other signals back to system controller 130, in addition to carrying a trigger signal from a trigger switch (not shown) associated with trigger button 216.

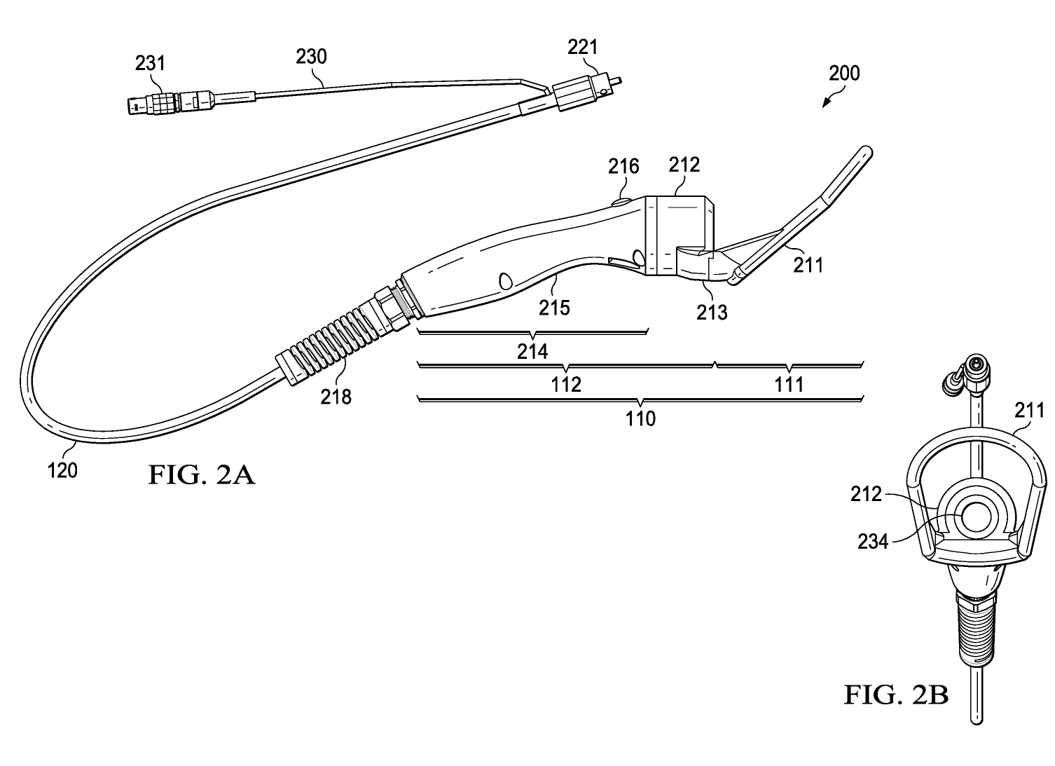

FIGS. 3A and 3B are side views of two exemplary laser handpieces designed to have two different bail 211 and attachment 213 structures for use at different treatment beam angles of incidence. Optical angle of incidence is measured conventionally from the normal to the target surface, i.e., away from perpendicular to the target surface. These two drawings include both handpiece 110 and the strain relief 218 part of a handpiece assembly 200, but omit details of umbilical 120 and associated optical and electrical connectors. In both of these examples, which have been fabricated as prototypes for testing on patients during real laser treatments, the overall scale is indicated by a diameter 48 mm of the distal portion 212 of housing 112, and other dimensions and angles have been labeled for discussion below. In both cases, the dimensions and angles are understood to be representative of purely exemplary embodiments, and are not to be construed as limiting the scope of the present invention.

In FIG. 3A, a handpiece configured to guide a user to hold it for an optical beam angle of incidence 301 set nominally to 50 degrees is illustrated. The spacing 303 from the front of distal portion 212 to the plane of the target surface (shown here as plane 311 coincident with a plane that during use is the bottom of bail 211) is set nominally at 52 mm. In use, as will be described later, a practitioner (user) grasps the handpiece, holding it by handle portion 214, and places bail 211 in substantial contact or proximity with a target surface (e.g. skin) of a patient to be treated, such that the bottom surface of bail 211, indicated by plane 311, is approximately parallel to the corresponding target surface on a patient. The bail 211 in this exemplary first design also comprises a forward portion 211′ that is slightly upturned at a small angle to facilitate repositioning and to reduce the bulging of skin toward the handpiece when applying usual pressure to the handpiece during use, which non-planar bulging deformation can affect the direction and pattern of backscattered laser power. In an exemplary second design, shown in FIG. 3B, the nominal angle of incidence 305 is set to 60 degrees, and spacing 307 from distal portion 212 to target surface plane 311 is nominally 65 mm. In this design, the bail 211 has a lower edge that falls along a single plane 311. Both of these designs shown in FIG. 3A and FIG. 3B implement spacings 303 and 307 as well as angles 301 and 305 that are within practical ranges that have been empirically found to mitigate handpiece heating due to backscattering from the target surface. A preferable range for spacings 303 and 307 is from approximately 50 to 75 mm, and a preferable range for angles of incidence 301 and 305 is from approximately 30 to 70 degrees.

FIGS. 4A and 4B show a more detailed view of an exemplary handpiece design that was illustrated previously in 2A, 2B, and 3A. Turning now to FIG. 4B, a cross-sectional view of the handpiece design is shown. Internal mechanical features that are used in attaching distal portion 212 to handle portion 214 are visible. Internal features of optics 114 and trigger 116 are also readily seen. While in some prior art laser handpieces, one or more lenses are used to shape a beam emerging from an optical fiber, the optical design shown in FIGS. 4A and 4B simply uses the divergence of an optical mode or beam from optical fiber 144 together with the spacing 303 to the target surface plane 311 (as previously shown in FIG. 3A) to determine the size and shape of a treatment beam spot on the target surface. Thus, in this design, the optics 114 referred to with respect to FIGS. 1A through 1D consist of free-space propagation together with optical window 412. An optics holder 410 within the distal portion 212 holds a fiber ferrule 414, and also supports optical window 412. Optics holder 410 can be made of a metal such as aluminum and machined for high precision in holding fiber ferrule 414 in accurate alignment with other optical elements such as lenses and windows. Metal also has good mechanical stability and heat dissipation properties for optics holder 410, should it be exposed to high scattered optical power. Distal portion 212 and handle portion 214 can be made of a different material than components of the optics holder 410, such as a thermoplastic polymer, e.g. acrylonitrile butadiene styrene (ABS), or other material that can be selected for ease of manufacturing curved ergonomic shapes such as through injection molding, and for possessing other useful properties like impact resistance, convenience in sterilization, etc.

Optical window 412 is used to seal the space between fiber end face 444 and window 412 in order to protect fiber end face 444 from dust, dirt, and other contaminants that are generated or collect during use or storage. Due to high optical power densities at fiber end face 444, such sealing is preferable in normal use, because contaminants on fiber end face 444 (which serves the role of emitting aperture 145 referred to previously) can be heated by exiting laser radiation and cause heating and catastrophic destruction of the fiber end. Window 412 can be anti-reflection coated at the wavelength(s) of light source 134 in order to minimize the optical power reflected from window 412 back into optics holder 410, and to maximize useful optical power transmitted through window 412 to the target surface to be treated.

Also visible in FIGS. 4A and 4B is trigger button 216, which can be seen connected to trigger switch 416 housed in handle portion 214. Electrical cable 146 can be seen connected to trigger switch 416, and joining optical fiber 144 to run alongside each other in a bundle within umbilical 120 (not shown, within strain relief 218). An auxiliary window 450 in handle portion 214 is provided in this embodiment for use by signal lights, displays, etc. In a prototype, a shock indicator that shows when the handpiece has been dropped, subjecting the handpiece to high G forces that can cause potential damage, has been mounted under auxiliary window 450. An exemplary suitable shock indicator for this purpose is the ShockWatch™ L30/100G impact indicator available from many vendors. Shock indicators having lower sensitivity (i.e., sensing higher accelerations) than 100G might also be suitable for determining whether possible damage has occurred to a handpiece.

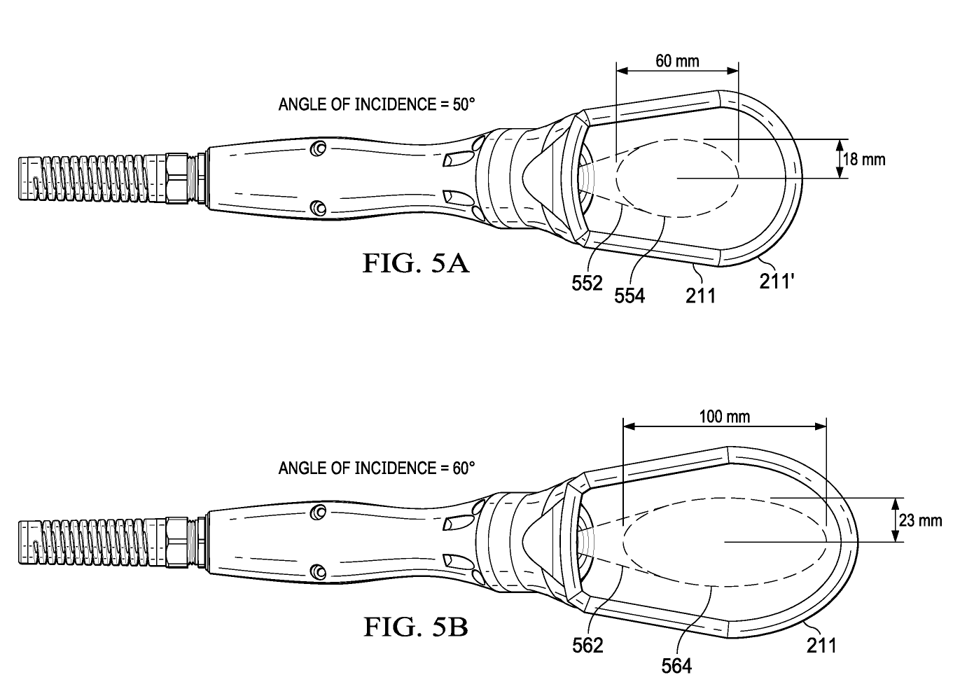

FIGS. 5A and 5B show views of the handpieces previously shown in FIGS. 3A (for 50° optical angle of incidence) and 3B (for 60° optical angle of incidence), respectively, from a direction perpendicular to plane 311, as if looking upward and outward from the area to be treated in a target surface. In FIG. 5A, it can be seen that for this combination of spacing 303 and a 50° angle of incidence 301, the diverging optical beam 552 illuminates a projected elliptical spot 554 in the plane 311 of the target surface to be treated (not shown) having half-width of 18 mm and a total length of 60 mm. Similarly, In FIG. 5B, for the combination of spacing 307 and 60° angle of incidence 305, the diverging optical beam 562 illuminates a projected elliptical spot 564 in the plane 311 of the target surface having a half-width of 23 mm and a total length of 100 mm.

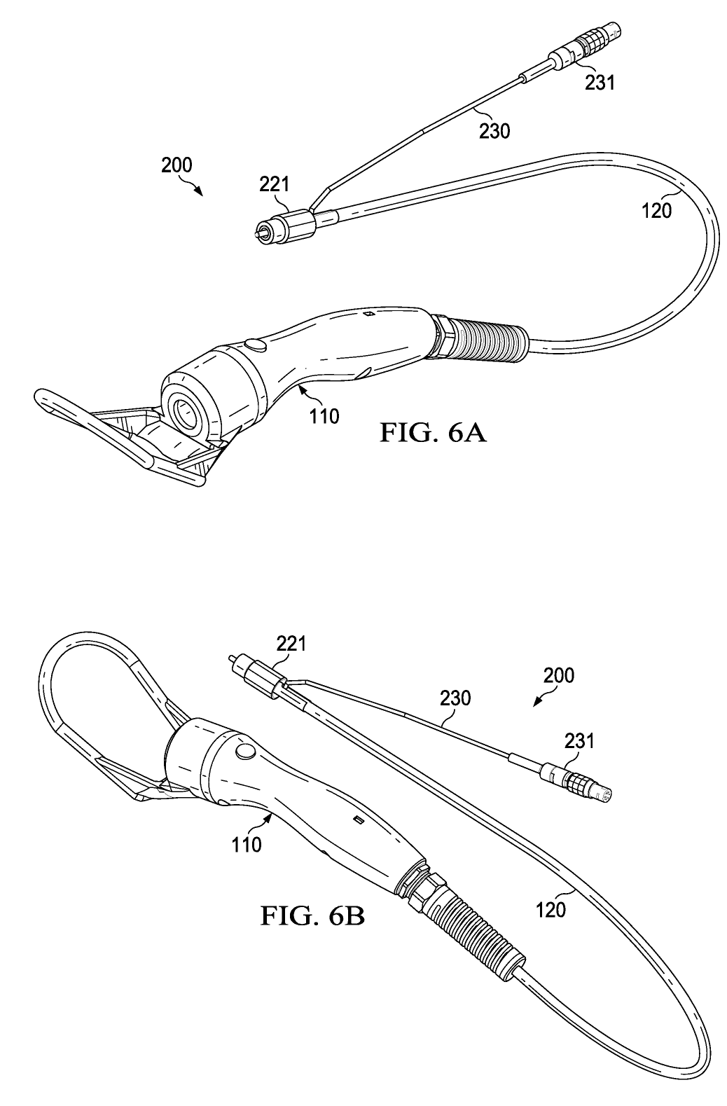

FIGS. 6A and 6B are line drawings showing two additional views of a laser handpiece assembly 200 similar to the design showed in FIG. 3A designed for a 50° angle of incidence.

Now referring to FIG. 7, a perspective line drawing of a prototype system for laser treatment 100 according to an embodiment of the present invention is shown. Handpiece 110 is connected through umbilical 120 containing an optical fiber 144 having a numerical aperture (NA) of 0.22 to system controller 130 containing a light source 134, that in this prototype comprises a 60-watt diode laser emitting at 810 nm wavelength. Lasers at other wavelengths such as 940 nm have also been employed. Suitable core and cladding diameters for optical fibers include core/cladding of 400/440 μm and 550/600 μm for use at these high laser powers. In other embodiments using different lasers, different optical fibers 144 can be used, but for withstanding high optical powers, large-core multimode optical fibers like these are preferable. Optical fiber connector 221 and connected electrical signal cable 230 are also indicated, showing how they are separately connected to system controller 130. These two connectors, namely fiber optic connector 221 and the connector on signal cable 230 (not shown, but hidden under a door on system controller 130), comprise part of the interface 131 for connecting handpiece 110 to system controller 130. Buttons, switches, knobs, and a display used for controlling system functions on controller 130 are shown, but not separately labeled.

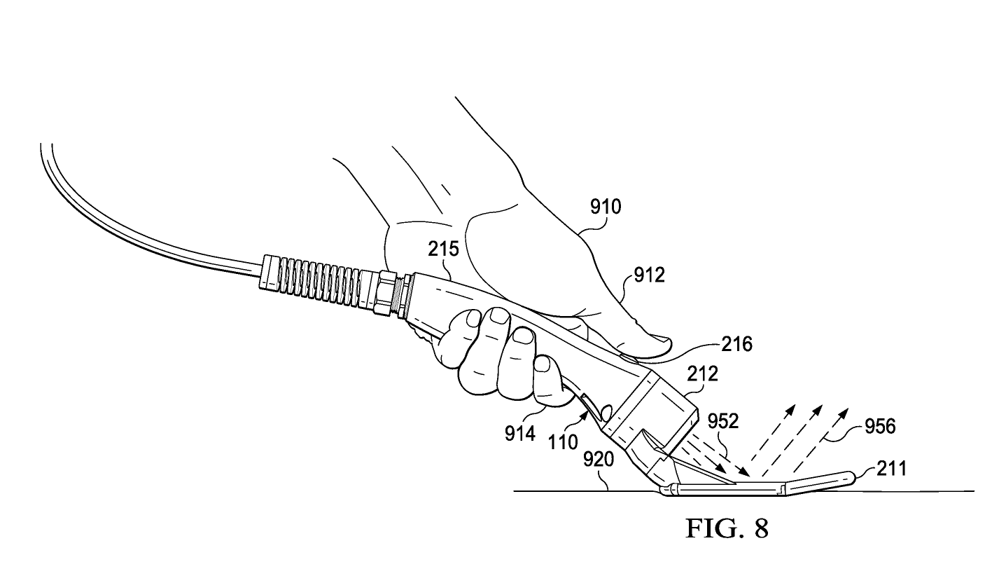

FIG. 8 is a side view drawing illustrating a method of use of a handpiece 110 according to an embodiment of the present invention. A practitioner's (user's) hand 910 grasps the handpiece grip 215 and the user places bail 211 in contact, substantial contact, or near-contact with a target surface 920 (e.g. skin) of a patient to be treated, so as to approximately align plane 311 (not shown in this FIG. 8) with the plane of target surface 920. The bail 211 is configured as described previously to place distal portion 212 in a proper position in terms of both spacing and angle to optimally irradiate target surface 920 when a user positions the handpiece and bail as described above. When user's thumb 912 (or other digit) is used to depress trigger button 216, treatment beam 952 is emitted incident upon target surface 920, and forward light beam 956 consisting of specular and forward-scattered light is indicated as in the drawing to continue diverging (as a result of the characteristics of chosen optics 114) along a forward path away from distal portion 212 of the handpiece 110. Thus, scattered light such as the forward beam 956 is preferentially directed away from the handpiece 110 and user's hand 910, rather than reflecting or scattering back toward distal portion 212 or hand 910, which could cause heating of the handpiece 110 or injury to the practitioner. In some embodiments (not shown), as described previously, a trigger button 216 or other feature(s) on grip 215 can be alternatively positioned and shaped so as to be conveniently operated by user's index finger 914, or another digit or combination of digits, e.g. using a squeezing motion. A combination of ergonomic and safety considerations may be used in the design of handpiece grip 215 and the shape and placement of trigger button 216, as well as the force required to operate the trigger, in order to optimize handpiece operation to minimize user fatigue, while simultaneously minimizing the chance of accidentally turning on the treatment laser beam when the handpiece is not in position.

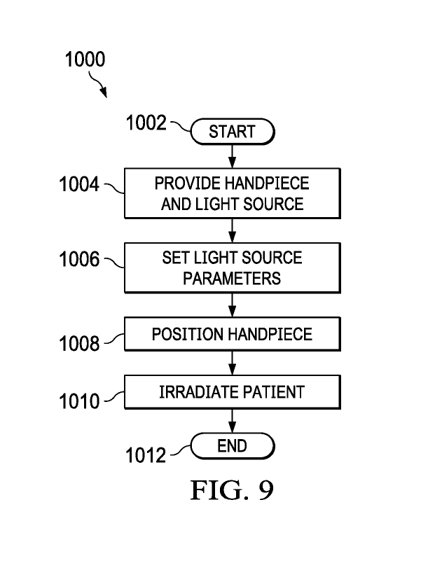

Referring now to FIG. 9, a flow chart of a method for laser treatment 1000 according to one embodiment of the present invention is shown. Treatment starts in step 1002; in step 1004, a light source is provided for use with the handpiece that has the power, wavelength, and pulsing capabilities required for a predetermined treatment protocol, and a handpiece is provided that has the features described herein for guiding the user in maintaining a proper angle and optionally a particular spacing of the handpiece from the target surface to be treated. In step 1006, the light source parameters are set for the laser treatment according to the predetermined treatment protocol. Next, the handpiece is positioned by the user in step 1008 with the standoff positioned so as to direct the light source (laser) beam at a predetermined angle (as aided by the design features of the standoff) and optionally a predetermined spacing from the target surface, and the handpiece is also positioned laterally to center the beam on a desired area of the target surface. Typically, the standoff 111 of the laser handpiece 110, in some embodiments comprising a bail 211, is placed in contact or near contact with the target surface as described above, which aids in accomplishing the proper positioning in angle and spacing. A visible aiming beam, as described earlier, may be used to help indicate the position on the target surface where the treatment beam will be incident. The patient is then irradiated in step 1010 while the handpiece is properly positioned as desired, by activating (switching on) the light source (e.g., laser) using, e.g., the trigger button 216 on the handpiece, or an alternative triggering device such as a footswitch described previously. Optional paths to return to earlier steps in the flow chart are not shown in FIG. 9, but one or more steps can be repeated, as follows: After possible repetition (not shown) of all of the steps 1004 through 1010, or only steps 1006 through 1010 if light source parameters need adjustment, or only steps 1008 through 1010 if only repositioning of the handpiece to multiple areas on the same patient is required, or if treatment of different patients can use the same light source parameters. After the patient or patients are irradiated, the method of treatment is ended at step 1012. The treatment protocol of FIG. 9 can be followed for different patients, or for different areas on one patient.

Although preferred embodiments of the present invention have been described in detail, it will be understood by those skilled in the art that various modifications can be made therein without departing from the spirit and scope of the invention. For example, instead of using a manually-held housing 112 having an ergonomic grip 215 to facilitate human grasping of handpiece 110, it is possible to use a machine or robot during treatment to hold and position the housing 112, which in turn can be of any convenient shape facilitating mechanical attachment and not constrained by ergonomics. In this case, bail 211, which is designed for human use in the example embodiments herein, could be replaced by a sensor or sensors that sense contact with the target surface, or that otherwise sense preferably both a distance from target surface 920 as well as an angle of incidence of light beam emitted from optical aperture 234 onto target surface 920, and transmit this sensed distance and/or angle to a processor that controls the robotic positioner, in order to maintain a distance and angle within prescribed ranges.

We claim:

1. A handpiece for laser treatment of a target surface, comprising:
a housing having an optical aperture;
an emitting aperture supported by the housing, from which treatment light emanates;
optics supported by the housing and configured to receive treatment light emanating from the emitting aperture and to direct the treatment light out the optical aperture toward the target surface; and
a standoff affixed to the housing and extending toward the target surface, configured to position the handpiece so that the treatment light is incident upon the target surface at a predetermined angle that is not perpendicular to the target surface.

2. The handpiece of claim 1, wherein the predetermined angle ranges from approximately 30 to approximately 70 degrees away from perpendicular to the target surface.

3. The handpiece of claim 1, wherein the standoff is a loop-shaped bail.

4. The handpiece of claim 1, wherein the standoff is further configured to set a distance of the handpiece to the target surface.

5. The handpiece of claim 1, wherein the standoff is detachable from the housing.

6. The handpiece of claim 1, wherein the housing is configured to be gripped by a user's hand.

7. The handpiece of claim 1, further comprising a trigger for turning the treatment light on and off.

8. The handpiece of claim 1, wherein the emitting aperture is an end face of an optical fiber that transmits light to the handpiece from a remote light source.

9. The handpiece of claim 1, further comprising a light source supported in the housing.

10. The handpiece of claim 9, wherein the light source is a laser, and the emitting aperture is an output aperture of the laser.

11. The handpiece of claim 1, further comprising a visible wavelength light source configured to produce an aiming beam indicating a position on the target surface toward which treatment light is directed.

12. The handpiece of claim 1, wherein the optics further comprise one or more optical elements selected from the group consisting of a window, a lens, a mirror, and a waveguide.

13. The handpiece of claim 1, wherein the optics further comprise a scanner.

14. The handpiece of claim 1, further comprising a means for cooling the target surface.

15. The handpiece of claim 1, further comprising a power supply contained within the housing.

16. A system for laser treatment of a target surface on a patient, comprising:
a system controller;
a light source in the system controller, capable of emitting treatment light;
a handpiece comprising a housing, an emitting aperture in the housing from which treatment light from the light source emanates, optics supported by the housing and configured to receive treatment light emanating from the emitting aperture and to direct the treatment light toward the target surface, and a standoff affixed to the housing and extending toward the target surface, the standoff configured to position the handpiece so that the treatment light is incident upon the target surface at a predetermined angle that is not perpendicular to the target surface; and
an umbilical connecting the handpiece to the system controller.

17. The system of claim 16, wherein the light source is not located in the system controller, but is instead located in the handpiece, and the emitting aperture is an aperture on the light source.

18. The system of claim 17, wherein there is no umbilical, and the system controller is contained within the handpiece.

19. A method for laser treatment comprising the steps of:
providing a light source capable of supplying treatment light, and a handpiece for directing the treatment light to a target surface on a patient, wherein the handpiece has a standoff configured to aid a user in directing the treatment light from the handpiece to be incident on the target surface at a predetermined angle that is not perpendicular to the target surface;
setting one or more parameters of the light source to predetermined values according to a treatment protocol;
positioning the handpiece so as to direct the treatment light at the predetermined angle toward an area of the target surface to be treated; and
activating the light source to irradiate the patient with the treatment light.

20. The method of claim 19, wherein the predetermined angle ranges from approximately 30 to approximately 70 degrees away from perpendicular to the target surface.
